(19) United States
(12) Patent Application Publication
(10) Pub. No.: US 2026/0111485 A1
(43) Pub. Date: Apr. 23, 2026

(54) SYSTEMS AND METHODS FOR INDEXING AND RETRIEVING ELECTRONIC INFORMATION

(71) Applicant: PAIGE.AI, Inc., New York, NY (US)

(72) Inventors: Sid SENTHILNATHAN, New York, NY (US); Jan BERNHARD, Brooklyn, NY (US)

(21) Appl. No.: 19/362,172

(22) Filed: Oct. 17, 2025

Related U.S. Application Data
(60) Provisional application No. 63/709,037, filed on Oct. 18, 2024.

(51) Int. Cl. G06F 16/901

(52) U.S. Cl. CPC G06F 16/901

(57) ABSTRACT

Described herein are systems and methods for indexing multimodal datasets including digital image data, structured textual data, unstructured textual data, keyword textual data, or any combination thereof. Further described herein are systems and methods for querying and searching indexed multimodal datasets to retrieve original source data relevant to the query. The systems and methods described herein are agnostic as to mode of data and as to mode of query.

### RELATED APPLICATIONS

This application claims priority to U.S. Provisional Patent Application No. 63/709,037 filed Oct. 18, 2024, which is hereby incorporated by reference in its entirety.

### FIELD OF THE DISCLOSURE

The present disclosure relates to systems and methods for processing medical information encompassing multiple data modalities using machine learning models.

### BACKGROUND

Analyzing medical information has traditionally been performed manually by medical professionals. Medical image analysis is central to the diagnosis and management of a broad spectrum of diseases, including oncology, infectious diseases, autoimmune and inflammatory conditions, genetic and metabolic disorders, neurological diseases, cardiovascular diseases, renal and liver diseases, pulmonary and dermatological conditions, hematologic and endocrine disorders, and transplant medicine. In clinical practice, images of pathology slides, radiology scans including MRI, CT, X-ray, ultrasound, PET, and SPECT, endoscopic images, and other specialized modalities are routinely examined to identify disease features that are visible within the image and to guide treatment decisions.

For a given patient, it may be helpful to compare a specific image to other images or other forms of data, from the same patient or from other patients, containing similar features. However, manually locating and reviewing such data can be challenging and time-consuming when searching across multiple modalities of medical data, such as digital images (e.g., whole slide images), molecular data (e.g., nucleic acid sequences), structured text (e.g., tabular data), and unstructured text (e.g., clinical notes). Medical data may also include image acquisition parameters, anatomical location and orientation, clinical indications, patient demographics, procedure details, image annotation and markups, quantitative image analysis results, interpretive reports, comparative references, quality control and validation data, linkages to other clinical or laboratory data, structured coding and classification, consent and regulatory information, workflow and provenance data, temporal data, device and software metadata, and external references. Thus, a multimodal medical dataset may encompass data originating from a wide range of imaging modalities and clinical sources, where each may have unique formats, resolutions, and metadata requirements, further complicating integration and analysis. Integrating multimodal data, such as combining imaging with genomic, laboratory, or clinical text data, presents additional challenges due to differences in data structure and semantics.

Additional factors may further complicate the process of integrating and analyzing multimodal medical data. Ensuring data privacy and security, maintaining data quality and completeness, and achieving interoperability and standardization across systems are significant concerns. Analyzing large medical datasets may be limited by annotation inconsistencies, variable tissue types within samples, very large image data sets, and other factors. Label scarcity and the need for expert annotation can limit the availability of high-quality training data, while class imbalance and rare events may impact model robustness. To capture the full diversity of complex domains, conventional models may require considerable parameter complexity, requiring extremely large datasets to train on. Training systems to analyze large, variable, and unannotated data may require vast amounts of computational power, particularly when the data includes high-resolution images such as those used in computational pathology. Additionally, analyzing temporal and longitudinal data or ensuring model interpretability for clinical use can further increase complexity.

Other challenges may include a lack of data, even when analysis of that data does not require exhaustive annotations. Even when utilizing supervised or weakly supervised training methods, the ability to generalize between applications may be limited, the availability of clinical labels or manual annotations may be reduced, and the training may generalize poorly with long tail distribution and rare events. The integration of data from diverse sources, such as different imaging modalities, laboratory results, genomic data, and clinical notes, can introduce additional complexity due to differences in data structure, format, and semantics. Ensuring interoperability and standardization across systems, maintaining data privacy and security, and addressing data quality and completeness are also significant concerns. The need for expert review and annotation, especially for specialized modalities or rare conditions, can further limit the scalability of training approaches. Conventional techniques fail to account for the challenges of analyzing large quantities of data across various modalities and without annotations.

### SUMMARY

Provided herein is a method for indexing multimodal datasets. The method may include receiving a multimodal dataset comprising a plurality of digital image inputs and a plurality of textual inputs; generating, using a group level aggregator model, a first plurality of group level vectors based on the plurality of digital image inputs; generating, using the group level aggregator model, a second plurality of slide level vectors based on the plurality of textual inputs; and storing the first plurality of slide level vectors and the second plurality of slide level vectors in an index.

In some aspects, generating the first plurality of group level vectors may involve generating, using a trained foundation model, a plurality of tile level vectors based on each digital image of the plurality digital image inputs; and aggregating, using the group level aggregator model, each plurality of tile level vectors to generate the first plurality of group level vectors. Each tile level vector may represent one or more features of interest extracted from individual regions within a corresponding digital image of the plurality of digital image inputs.

Foundation models may mean models trained on large-scale multimodal data, that are usable for a wide range, including multimodal, purposes. As used herein, the term foundation model may correspond to a single model, or it may correspond to any combination of algorithms, machine learning models, artificial intelligence models, or other logic. In at least one aspect, for example, the embedding generation may be performed by the trained foundation model, but other aspects such as the group or vector segmentation, region of interest identification, etc., might not be performed by the trained foundation model. The techniques discussed herein may involve combinations of algorithms, rules, logic, and/or models that may operate in tandem with the trained foundation model to affect the implementations discussed herein.

In some aspects, the plurality of digital image inputs may include digital medical images, such as an image of a cytology specimen, an image of histopathology specimen, a whole slide image, a multiplex immunofluorescent image, a multiplex immunohistochemistry image, a magnetic resonance imaging (MRI) image, a computed tomography (CT) image, an X-ray image, a nuclear medicine imaging (NMI) image, an ultrasound image, a mammography image, an endoscopic image, an angiography image, a confocal microscopy image, a fluorescence in situ hybridization image, an optical coherence tomography image, a bone scan image, a thermography image, an electron microscopy image, and/or other images supporting detailed visualization and characterization of tissue specimens to evaluate disease mechanisms, progression, and therapeutic response across diverse clinical contexts.

In some aspects, the plurality of textual inputs may include unstructured text, keyword text, structured text, or a combination thereof. Unstructured text may include diagnosis information, notes regarding sample retrieval and/or preparation, histological details, clinical context involving patient history and other modalities of tests, information specific to staining and markers, morphological observations, transcripts from auditory comments or opinions, references to other tests, references to treatment data, or a combination thereof. Keyword text may include a medical term, a diagnostic code, or a morphological descriptor. Structured text may include tabular data, genetic sequencing data, genomic data, molecular data, proteomic data, standardized diagnostic reports, coded medical records, or templated clinical forms.

In some aspects, generating the first plurality of slide level vectors and/or the second plurality of slide level vectors may include aggregation, or aggregation and compression.

Further provided herein are systems for indexing multimodal datasets. The system may include at least one memory storing instructions, and at least one processor configured to execute the instructions to perform operations. The operations may include receiving a multimodal dataset comprising a plurality of digital image inputs and a plurality of textual inputs; generating, using a group level aggregator model, a first plurality of group level vectors based on the plurality of digital image inputs; generating, using the group level aggregator model, a second plurality of slide level vectors based on the plurality of textual inputs; and storing the first plurality of slide level vectors and the second plurality of slide level vectors in an index.

In some aspects, generating the first plurality of group level vectors may involve generating, using a trained foundation model, a plurality of tile level vectors based on each digital image of the plurality digital image inputs; and aggregating, using the group level aggregator model, each plurality of tile level vectors to generate the first plurality of group level vectors. Each tile level vector may represent one or more features of interest extracted from individual regions within a corresponding digital image of the plurality of digital image inputs.

In some aspects, the plurality of digital image inputs may include digital medical images, such as an image of a cytology specimen, an image of histopathology specimen, a whole slide image, a multiplex immunofluorescent image, a multiplex immunohistochemistry image, a magnetic resonance imaging (MRI) image, a computed tomography (CT) image, an X-ray image, a nuclear medicine imaging (NMI) image, an ultrasound image, a mammography image, an endoscopic image, an angiography image, a confocal microscopy image, a fluorescence in situ hybridization image, an optical coherence tomography image, a bone scan image, a thermography image, an electron microscopy image, and/or other images supporting detailed visualization and characterization of tissue specimens to evaluate disease mechanisms, progression, and therapeutic response across diverse clinical contexts.

In some aspects, the plurality of textual inputs may include unstructured text, keyword text, structured text, or a combination thereof. Unstructured text may include diagnosis information, notes regarding sample retrieval and/or preparation, histological details, clinical context involving patient history and other modalities of tests, information specific to staining and markers, morphological observations, transcripts from auditory comments or opinions, references to other tests, references to treatment data, or a combination thereof. Keyword text may include a medical term, a diagnostic code, or a morphological descriptor. Structured text may include tabular data, genetic sequencing data, genomic data, molecular data, proteomic data, standardized diagnostic reports, coded medical records, or templated clinical forms.

In some aspects, generating the first plurality of slide level vectors and/or the second plurality of slide level vectors may include aggregation, or aggregation and compression.

Further provided herein is a non-transitory computer-readable medium storing instructions that, when executed by a processor, cause the processor to perform a method for indexing a multimodal dataset. The method may include receiving a multimodal dataset comprising a plurality of digital image inputs and a plurality of textual inputs; generating, using a group level aggregator model, a first plurality of group level vectors based on the plurality of digital image inputs; generating, using the group level aggregator model, a second plurality of slide level vectors based on the plurality of textual inputs; and storing the first plurality of slide level vectors and the second plurality of slide level vectors in an index.

In some aspects, generating the first plurality of group level vectors may involve generating, using a trained foundation model, a plurality of tile level vectors based on each digital image of the plurality digital image inputs; and aggregating, using the group level aggregator model, each plurality of tile level vectors to generate the first plurality of group level vectors. Each tile level vector may represent one or more features of interest extracted from individual regions within a corresponding digital image of the plurality of digital image inputs.

In some aspects, the plurality of digital image inputs may include digital medical images, such as an image of a cytology specimen, an image of histopathology specimen, a whole slide image, a multiplex immunofluorescent image, a multiplex immunohistochemistry image, a magnetic resonance imaging (MRI) image, a computed tomography (CT) image, an X-ray image, a nuclear medicine imaging (NMI) image, an ultrasound image, a mammography image, an endoscopic image, an angiography image, a confocal microscopy image, a fluorescence in situ hybridization image, an optical coherence tomography image, a bone scan image, a thermography image, an electron microscopy image, and/or other images supporting detailed visualization and characterization of tissue specimens to evaluate disease mechanisms, progression, and therapeutic response across diverse clinical contexts.

In some aspects, the plurality of textual inputs may include unstructured text, keyword text, structured text, or a combination thereof. Unstructured text may include diagnosis information, notes regarding sample retrieval and/or preparation, histological details, clinical context involving patient history and other modalities of tests, information specific to staining and markers, morphological observations, transcripts from auditory comments or opinions, references to other tests, references to treatment data, or a combination thereof. Keyword text may include a medical term, a diagnostic code, or a morphological descriptor. Structured text may include tabular data, genetic sequencing data, genomic data, molecular data, proteomic data, standardized diagnostic reports, coded medical records, or templated clinical forms.

In some aspects, generating the first plurality of slide level vectors and/or the second plurality of slide level vectors may include aggregation, or aggregation and compression.

### BRIEF DESCRIPTION OF DRAWINGS

Non-limiting and non-exhaustive examples are described with reference to the following figures.

FIG. 1 is a diagram illustrating an exemplary system, according to aspects of the present disclosure.

FIG. 2 is a diagram illustrating an exemplary workflow for indexing a dataset, according to aspects of the present disclosure.

FIG. 3A is a diagram illustrating exemplary methods for indexing a dataset, according to aspects of the present disclosure.

FIG. 3B is a diagram illustrating exemplary methods for indexing a dataset, according to aspects of the present disclosure.

FIG. 4 is a diagram illustrating a workflow for querying and searching a multimodal dataset, according to aspects of the present disclosure.

FIG. 5 is a diagram illustrating exemplary methods for querying and searching a multimodal dataset, according to aspects of the present disclosure.

FIG. 6 is a diagram illustrating an exemplary computing device, according to aspects of the present disclosure.

### DETAILED DESCRIPTION OF THE EMBODIMENTS

The following description sets forth exemplary aspects of the present disclosure. It should be recognized, however, that such description is not intended as a limitation on the scope of the present disclosure. Rather, the description also encompasses combinations and modifications to those exemplary aspects described herein.

Various embodiments described herein relate to systems and methods for indexing, querying, and searching patient information across multiple modalities in pathology applications. Powered by machine learning models, the systems and methods process and analyze diverse forms of medical data, including digital pathology images, natural language text, genomic information, and other clinical data types. In some cases, the systems may generate vector representations of patient information that enable efficient searching and retrieval of relevant medical data based on query inputs provided by users. In some cases, a user may search for pathology information using a digital image as a query input, while in other cases, the same system may process text queries to retrieve similar or related pathology data. The vector-based approach may facilitate rapid comparison and matching of diverse data types within large pathology datasets.

The disclosed system demonstrates an improvement to existing technology by overcoming the limitations of conventional pathology data analysis methods that require manual searching and are constrained by inconsistent data formats and natural language variations. Unlike traditional approaches that struggle with large, variable, and unannotated datasets across multiple modalities, this machine learning-powered system can index and search thousands of digital pathology images and associated metadata regardless of input format. The modality-agnostic capability represents a significant advancement over existing systems that typically require specific input formats, as it allows users to search using either image inputs or text inputs through the same unified tool, thereby eliminating the need for data conversion and enabling more efficient and comprehensive pathology dataset analysis.

The systems and methods may be implemented across various pathology applications and use cases, including the following examples. The system may facilitate research studies or clinical trials by enabling researchers to quickly locate relevant pathology data within large clinical trial datasets. The system may support identification of rare pathology features by comparing novel or unusual cases against extensive databases of indexed pathology information. The system may assist in misdiagnosis identification by enabling comparison of current cases with similar historical cases and their associated diagnostic outcomes. The system may implement individual patient dataset indexing by organizing and indexing pathology information specific to an individual patient and/or their family. The system may enable identification of textbook examples of known morphologies by matching query inputs against well-characterized pathology cases. A healthcare institution may utilize the systems to suggest similar cases within the same institution or site as references for clinicians to review during diagnostic processes. The system may perform screening functions by conducting low-cost vector lookups to assess likelihood of certain cancers or biomarkers, potentially reducing computational costs compared to more resource-intensive artificial intelligence inference processes. Clinical trial enrollment acceleration may be supported through the system's ability to rapidly identify patients or cases meeting specific criteria based on pathology characteristics.

The system may streamline molecular testing workflows by flagging slides and tissue blocks that may be suitable as source tissue for next generation sequencing panels. The system may improve efficiency of pathology review workflows by ranking slides for review priority within individual cases. The system may be used in a dataset curation service that retrieves relevant or the most relevant records within an indexed dataset based on a small number of sample prototypes across data modalities (language, image, genetic sequences, etc.) and based on a request of desired sample counts of the fully curated dataset, and captures variance represented by the sample prototypes, thereby accelerating the curation of training datasets and evaluation datasets.

The system may be implemented to verify if given input data satisfies semantic content constraints (e.g., surgical sample collection method, tissue type, tumor origin, etc.), thereby verifying the content to be appropriate for downstream processing. For example, the system may receive a customer request to run a prostate clinical model on an image of breast tissue, based on which the system may identify and/or output that the image is out-of-distribution for the intended use of the prostate model. As another example, knowing the distribution of valid input embeddings, the system may direct the input to the desired downstream services without explicitly requiring additional data or metadata.

These diverse applications demonstrate the flexibility and broad applicability of the machine learning-based indexing and retrieval systems across various pathology and clinical contexts.

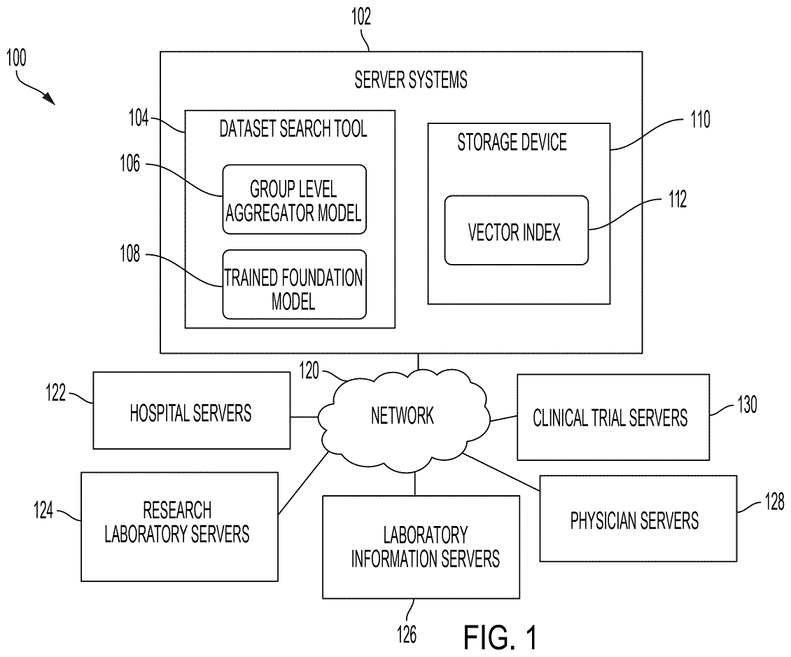

FIG. 1 depicts an exemplary system 100 for retrieving digital pathology images based on query inputs across distributed healthcare environments. System 100 may include server systems 102 that may serve as a central processing hub for pathology data analysis and retrieval operations. Server systems 102 may include a dataset search tool 104 configured to process and analyze various forms of medical data received from multiple sources. Dataset search tool 104 may utilize machine learning capabilities to enable cross-modal searching and vector-based data retrieval across diverse pathology datasets. The distributed architecture may facilitate comprehensive data collection by connecting multiple healthcare institutions and research facilities through standardized communication protocols.

Dataset search tool 104 may include a group level aggregator model 106 and a trained foundation model 108 or other machine learning model that work together to process medical data information. Group level aggregator model 106 may be configured to combine and compress vector representations generated from different data sources and modalities, enabling efficient storage and retrieval of medical data at various hierarchical levels. For example, group level aggregator model 106 may be configured to receive textual inputs, digital image inputs, and/or embeddings representing features of textual inputs or digital image inputs, and may aggregate the inputs to generate one or more group level vectors. The one or more group level vectors may include embeddings representing a feature of interest at different organizational levels, including individual areas of focus within whole slide images, complete whole slide images, collections of multiple whole slide images, tissue blocks, organs, bones, bodily structures, individual patients, or populations of patients. The one or more group level vectors may also include embeddings representing a feature of interest across various modalities of medical data, such representation of a feature of interest within a digital medical image and within a genetic sequence. Optionally, group level aggregator model 106 may be configured to perform compression to compress the one or more group level vectors.

In some embodiments, group level aggregator model 106 may be further configured to generate a vector index containing the one or more group level vectors, which may be stored for future uses, such as indexing, similarity searching, cross-modal and cross-patient retrieval, clinical research applications, dataset curation, model validation, routing, or anomaly detection, continuous learning and updating, impression and storage efficiency, etc., as discussed herein. As described further below, dataset search tool 104 may be configured to search the vector index based on a query input to determine the most relevant search results based on similarity scores. The vector index may hold a “snapshot” of the embeddings encoding the aspects of the data it is linked to, which may enable the downstream uses discussed herein. The vector index may be regularly or continuously updated with new group level vectors generated by the systems and networks described herein. In some embodiments, group level vectors may be split across multiple vector indexes, all mapping to the same patient information source. Dataset search tool 106 may search the multiple vector indexes based on the query input and determine the most relevant search results by reducing similarity scores across the multiple vector indexes to intermediate values of the aggregation, a single similarity score, etc.

In some embodiments, group level aggregator model 106 may be configured to receive one or more embeddings from trained foundation model 108. Trained foundation model 108 may be trained on extensive medical datasets to recognize patterns and features across different imaging modalities and associated clinical information. The training data may include thousands or millions of digital medical images, encompassing samples from a wide variety of organs, multiple types of samples (i.e., biopsy, resection, aspiration, etc.), and the long tail of rare disease states and subtypes. Trained foundation model 108 may also be trained on data associated with digital medical images, encompassing multiple modalities of data as described herein. In some aspects, trained foundation model 108 may include a masked autoencoder (MAE) model, a distilled MAE model, or a hierarchical MAE model, a contrastive learning model, a transformer-based self-supervised model (non-MAE), a diffusion or variational autoencoder generative model, a cross-modal or joint embedding multimodal encoder, a masked language model or multimodal transformer, and/or a graph neural network of hierarchical aggregator.

Trained foundation model 108 may be configured to receive one or more medical images and a query to generate one or more embeddings from the one or more medical images. The one or more embeddings may include tile level vectors representing inferred features within the one or more medical images. The one or more medical images may include pathology slide images, radiology scan images including MRI, CT, X-ray, ultrasound, PET, and SPECT, and more. For example, the one or more medical images may include an image of a cytology specimen, an image of histopathology specimen, a whole slide image, a multiplex immunofluorescent image, a multiplex immunohistochemistry image, a magnetic resonance imaging (MRI) image, a computed tomography (CT) image, an X-ray image, a nuclear medicine imaging (NMI) image, an ultrasound image, a mammography image, an endoscopic image, an angiography image, a confocal microscopy image, a fluorescence in situ hybridization image, an optical coherence tomography image, a bone scan image, a thermography image, an electron microscopy image, or other images supporting detailed visualization and characterization of tissue specimens to evaluate disease mechanisms, progression, and therapeutic response across diverse clinical contexts. Upon receiving one or more embeddings from trained foundation model 108, group level aggregator model 106 may generate one or more group level vectors including one or more embeddings representing an entire image.

Server systems 102 may further include storage device 110 configured to maintain indexed pathology information for rapid retrieval and analysis. Storage device 110 may contain a vector index 112 storing vector representations of pathology data in a format that enables efficient similarity-based searching and matching operations. Vector index 112 may organize pathology information according to the hierarchical levels established by slide level aggregator model 106, allowing users to search for similar cases at appropriate levels of granularity. In some cases, vector index 112 may be updated continuously or at regular intervals as new pathology data becomes available from connected healthcare facilities and research institutions.

Server systems 102 may be connected to a network 120 that enables communication with various external healthcare and research facilities. Network 120 may comprise electronic communication infrastructure such as the internet, private networks, or specialized medical data networks that facilitate secure transmission of pathology information between institutions. The distributed architecture may enable the system to collect and process pathology data from diverse sources while maintaining appropriate security and privacy protections for sensitive medical information. Network 120 may support various communication protocols and data formats to accommodate different institutional systems and data management approaches.

Network 120 may connect server systems 102 to hospital servers 122, research laboratory servers 124, laboratory information servers 126, physician servers 128, and clinical trial servers 130. Hospital servers 122 may provide access to clinical pathology data generated during routine patient care activities, including digital slide images, diagnostic reports, and associated clinical information. Research laboratory servers 124 may contribute specialized research data, experimental results, and advanced imaging data that may enhance the comprehensiveness of the pathology dataset. Laboratory information servers 126 may contain structured laboratory data, test results, and standardized pathology reports that provide additional context for image-based pathology information. Physician servers 128 may supply clinical observations, diagnostic interpretations, and treatment-related information that may be valuable for comprehensive pathology analysis. Clinical trial servers 130 may contribute research data from controlled studies, enabling the system to incorporate evidence-based pathology information and treatment outcome data into the searchable dataset.

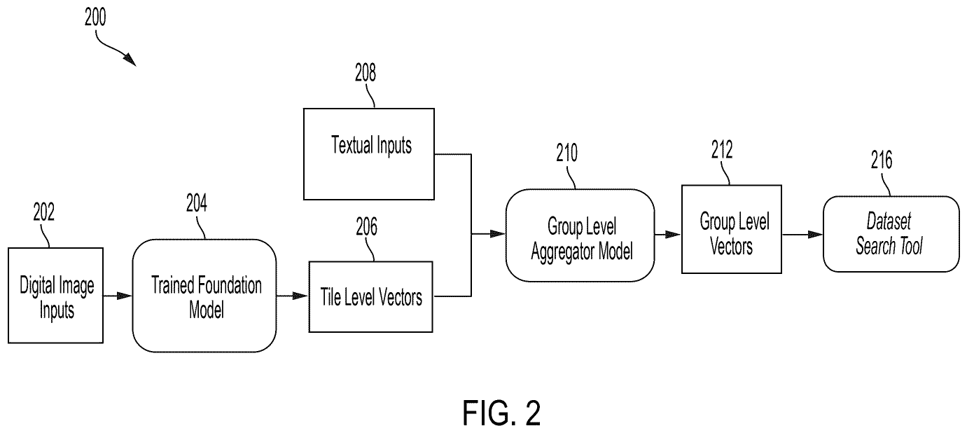

FIG. 2 depicts an exemplary workflow 200 for generating group level vectors from medical datasets. Workflow 200 may include receiving multiple types of input data and processing the input data through sequential machine learning models to generate hierarchical vector representations suitable for indexing and searching operations. The systematic approach may enable dataset search tool 104 to handle various data modalities while maintaining consistent vector-based representations that facilitate cross-modal searching capabilities. Diverse imaging modalities may enable workflow 200 to process comprehensive medical datasets that span multiple diagnostic approaches and imaging technologies.

Workflow 200 may include receiving digital image inputs 202 encompassing various medical imaging modalities used in medical practice. Digital image inputs 202 may include pathology slide images, radiology scan images including MRI, CT, X-ray, ultrasound, PET, and SPECT, and more. For example, digital image inputs 202 may include an image of a cytology specimen, an image of histopathology specimen, a whole slide image, a multiplex immunofluorescent image, a multiplex immunohistochemistry image, a magnetic resonance imaging (MRI) image, a computed tomography (CT) image, an X-ray image, a nuclear medicine imaging (NMI) image, an ultrasound image, a mammography image, an endoscopic image, an angiography image, a confocal microscopy image, a fluorescence in situ hybridization image, an optical coherence tomography image, a bone scan image, a thermography image, an electron microscopy image, or other images supporting detailed visualization and characterization of tissue specimens to evaluate disease mechanisms, progression, and therapeutic response across diverse clinical contexts. In some embodiments, digital image inputs 202 may be received from hospital servers 122, research laboratory servers 124, laboratory information servers 126, physician servers 128, and/or clinical trial servers 130 through network 120.

Digital image inputs 202 may be processed by a trained foundation model 204 that generates vector representations of the image content. Trained foundation model 204 may correspond to trained foundation model 108 within dataset search tool 104 and may be trained on extensive medical datasets to recognize patterns, morphological features, and biomarkers across different tissue types and pathological conditions. In some embodiments, trained foundation model 204 may process individual portions, tiles, or region within a medical image to generate one or more tile level vectors representing local features or characteristics. In other embodiments, trained foundation model 204 may process an entire image to generate one or more tile level vectors representing local features or characteristics. Tile level vectors 206 may capture local morphological characteristics, cellular patterns, tissue architecture, and other pathologically relevant features that may be present within specific areas of pathology images. Trained foundation model 204 may generate tens, hundreds, thousands, or more tile level vectors 206 per image, depending on the image size and the level of detail required for analysis.

Workflow 200 may also include receiving textual inputs 208 providing medical information and clinical observations. Textual inputs 208 may include unstructured text, keyword text, structured text, or a combination thereof.

As used herein, unstructured text refers to various forms of natural language writing, such as freeform writing. Unstructured text may include tabular medical data, diagnosis information, notes regarding sample retrieval and/or preparation, specific histological details, clinical context involving patient history and other modalities of tests, information specific to the staining and markers, morphological observations, transcripts from auditory comments or opinions, references to other tests, references to treatment data, or any combination thereof.

As used herein, keyword text refers to specific terms or phrases used to identify, categorize, or search for particular concepts, topics, or content within a dataset. Unlike natural language text, keyword text consists of concise, targeted words or short phrases that serve as labels or tags to describe key attributes or characteristics of the associated data. Keyword text may include medical terminology, diagnostic codes, or specific morphological descriptors that facilitate efficient indexing and retrieval of relevant patient information.

As used herein, structured text refers to textual information that is organized according to a predefined format, schema, or set of rules, making it machine-readable and easily processable. This type of text typically follows consistent patterns, uses standardized fields or categories, and maintains uniform formatting conventions that enable systematic data extraction and analysis. Structured text may include genetic sequencing data (e.g., nucleic acid sequences or amino acid sequences), genomic data, molecular data, proteomic data, standardized diagnostic reports, coded medical records, or templated clinical forms that contain information organized in specific sections or data fields. Genetic sequencing data, genomic data, molecular data, and proteomic data may provide additional context for pathology analysis and searching operations. For example, genetic sequencing data (e.g., nucleic acid sequences or amino acid sequences) and genomic data (e.g., gene expression information, genomic variants, tumor sequencing data, protein expression levels, and non-coding RNA expression levels) may provide biochemical information that may complement morphological observations from pathology images and clinical descriptions. Textual inputs 208 may provide valuable context that may enhance the searchability and clinical relevance of the indexed medical data. Textual inputs 208 may be obtained from pathologists or medical professionals, laboratory servers, patients, or by another machine learning model or artificial intelligence (AI) agent that may process and summarize clinical information.

Workflow 200 may include processing tile level vectors 206 and textual inputs 208 using a group level aggregator model 210 that generates group level vectors 212 representing joint vector representations. Group level aggregator model 210 may correspond to group level aggregator model 106 within dataset search tool 104. Group level aggregator model 210 may include a foundation model trained on extensive medical datasets spanning diverse tissue types. The foundation model may include an aggregator architecture configured to generate a joint representation across any modality of data, such as tile embeddings, natural language, nucleic acid sequences, tabular medical data, and more.

In some aspects, group level aggregator model 210 may perform aggregation operations that combine multiple tile level vectors 206 from individual images to generate group level vectors 212 including comprehensive representations of entire images. Group level aggregator model 210 may also incorporate textual inputs 208 during the aggregation process such that group level vectors 212 are multimodal vector representations combining image-based and text-based information. In this manner, workflow 200 generates compact vector representations that maintain relevant pathological information while reducing computational and storage requirements. Accordingly, the group level vectors 212 generated by group level aggregator model 210 are a more concise vector representation of a plurality of sub-group vectors and/or multimodal inputs than the sub-group vectors or tile level vectors themselves. Further, group level vectors 212 represent medical information at higher hierarchical levels compared to tile level vectors 206. The hierarchical vector approach facilitates efficient searching and retrieval operations across large pathology datasets while maintaining appropriate levels of detail for diagnostic and research applications.

Workflow 200 may further include providing slide level vectors 212 to a dataset search tool 216 that may index and store the group level vectors 212 for subsequent searching and retrieval operations. Dataset search tool 216 may correspond to dataset search tool 104 within server systems 102 and may be configured to organize and maintain indexed pathology information in vector index 112. In some aspects, dataset search tool 216 may include a fully managed vector database solution (e.g., Azure AI Search, Pinecone, MongoDB), a self-hosted database (e.g., Milvus or Qdrant), or an open-source library (e.g., DocArray or FAISS). Dataset search tool 216 may store slide level vectors 212 in a format that enables efficient similarity-based searching and matching operations when users provide query inputs. The indexing process may organize pathology information according to vector similarity measures that may facilitate rapid identification of related or similar pathology cases. Dataset search tool 216 may also support continuous or regular updates to the indexed information as new pathology data becomes available from connected healthcare facilities and research institutions through network 120.

### Indexing

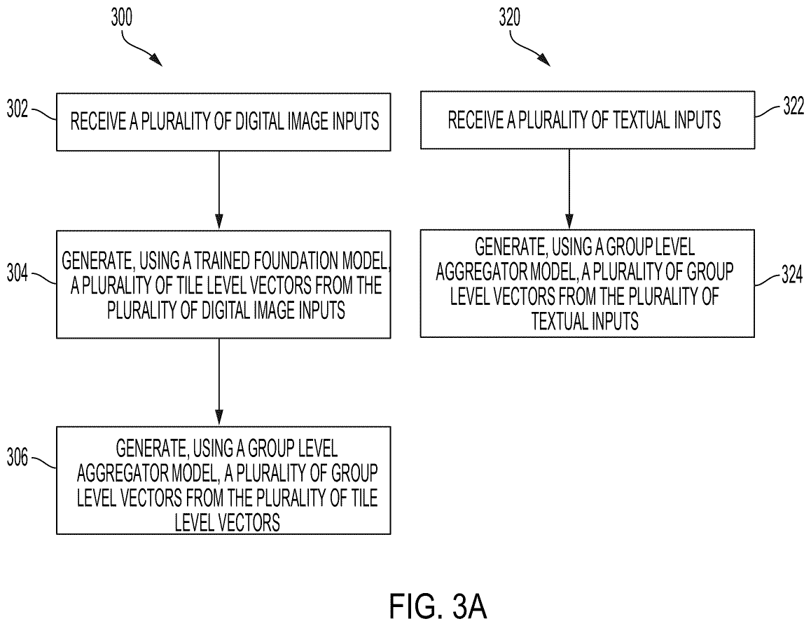

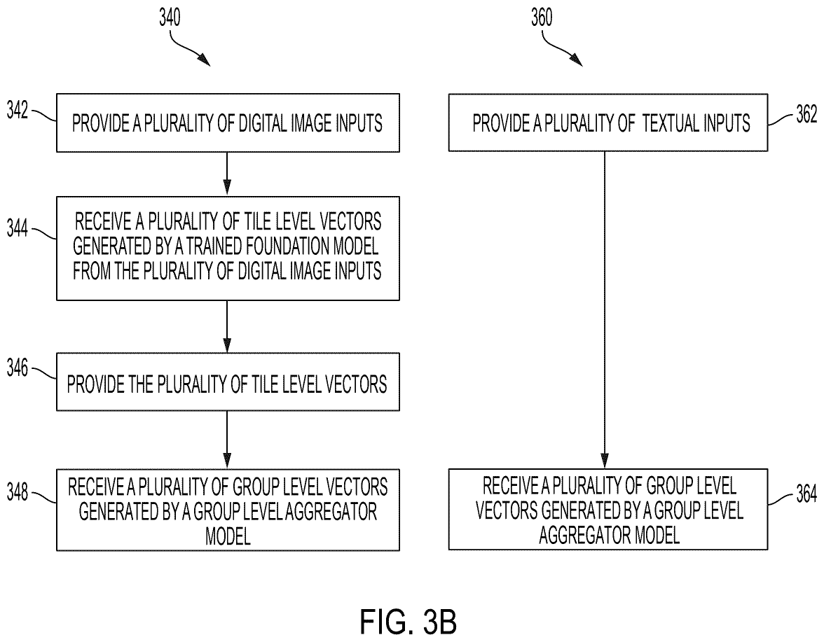

FIGS. 3A and 3B depict exemplary methods for indexing a pathology dataset from digital image inputs, textual inputs, or a combination thereof. In this manner, the systems and networks described herein are capable of combining modalities of patient information beyond embeddings of a single multi-modal model (such as a single model for digital slide images and textual inputs).

FIG. 3A depicts a method 300 for indexing digital medical images. Method 300 may include step 302 of receiving a plurality of digital image inputs from hospital servers 122, research laboratory servers 124, laboratory information servers 126, physician servers 128, clinical trial servers 130, or any combination thereof. The plurality of digital image inputs may correspond to digital image inputs 202.

Method 300 may further include step 304 of generating, using a trained foundation model, a plurality of tile level vectors from the plurality of digital image inputs. The trained foundation model may correspond to trained foundation model 108, 204. The plurality of tile level vectors may correspond to plurality of tile level vectors 206.

Method 300 may further include step 306 of generating, using a group level aggregator model, a plurality of group level vectors from the plurality of tile level vectors. The group level aggregator model may correspond to group level aggregator model 106, 210, and the plurality of group level vectors may correspond to group level vectors 212. In some aspects, generating the plurality of group level vectors may include performing aggregation. In some aspects, generating the plurality of group level vectors may include performing aggregation and compression. In some aspects, generating the plurality of group level vectors may include generating a vector index, which may be stored for future uses, such as indexing, similarity searching, cross-modal and cross-patient retrieval, clinical research applications, dataset curation, model validation, routing, or anomaly detection, continuous learning and updating, impression and storage efficiency, etc., as discussed herein. In some aspects, each group level vector is a joint vector representation/joint embedding/aggregated multimodal embedding that captures features of an entire image.

FIG. 3A further depicts a method 320 for indexing textual medical data. Method 320 may include step 322 of receiving a plurality of textual inputs from hospital servers 122, research laboratory servers 124, laboratory information servers 126, physician servers 128, clinical trial servers 130, or any combination thereof. The plurality of textual inputs may correspond to plurality of textual inputs 208. In some aspects, the plurality of textual inputs may include unstructured text, keyword text, structured text, or a combination thereof, as described herein above. In some aspects, the plurality of textual inputs may be obtained from a pathologist, a medical professional, or a patient, or from another machine learning model or artificial intelligence (AI) agent in a workflow.

Method 320 may further include step 324 of generating, using a group level aggregator model, a plurality of slide level vectors from the plurality of textual inputs. The group level aggregator model may correspond to group level aggregator model 106, 210, and the plurality of group level vectors may correspond to group level vectors 212. In some aspects, generating the plurality of group level vectors may include performing aggregation. In some aspects, generating the plurality of group level vectors may include performing aggregation and compression. In some aspects, generating the plurality of group level vectors may include generating a vector index containing the plurality of group level vectors in a form suitable for storage and further uses. The vector index may be regularly or continuously updated with new group level vectors generated by the systems and networks described herein. Group level vectors may be split across multiple vector indexes, all mapping to the same patient information source.

The steps of method 300 and method 320 may be combined into a method for indexing a medical dataset including both digital image inputs and textual inputs. The group level aggregator model may perform step 306 to generate group level vectors representing one or more features of interest in entire images, and the same group level aggregator model may perform step 324 to generate group level vectors representing one or more features of interest in textual inputs. The group level aggregator model may also combine vectors representing features of interest from digital image inputs with vectors representing textual inputs that provide data associated with the digital image inputs.

FIG. 3B depicts a method 340 for indexing digital medical images. Method 340 may include step 342 of providing a plurality of digital image inputs obtained from a user, such as a pathologist, a medical professional, a patient, or a service provider, or by another machine learning model or artificial intelligence (AI) agent as part of a workflow. The plurality of digital image inputs may correspond to digital image inputs 202.

Method 340 may further include step 344 of receiving a plurality of tile level vectors generated by foundation model from plurality of digital image inputs. The foundation model may correspond to trained foundation model 108, 204, and the plurality of tile level vectors may correspond to tile level vectors 206.

Method 340 may further include step 346 of providing the plurality of tile level vectors to a group level aggregator model. The group level aggregator model may correspond to group level aggregator model 106, 210. The plurality of tile level vectors may be provided by a user, such as a medical professional, or by another machine learning model or artificial intelligence (AI) agent as part of a workflow.

Method 340 may further include step 348 of receiving a plurality of group level vectors generated by the group level aggregator model. The plurality of group level vectors may correspond to group level vectors 212. In some aspects, the group level aggregator model may generate the plurality of group level vectors by performing aggregation. In some aspects, the group level aggregator model may generate the plurality of group level vectors by performing aggregation and compression. In some aspects, the group level aggregator model may generate the plurality of group level vectors in the form of a vector index containing the plurality of group level vectors in a form suitable for storage and further uses. The vector index may be regularly or continuously updated with new group level vectors generated by the systems and networks described herein. Group level vectors may be split across multiple vector indexes, all mapping to the same patient information source.

FIG. 3B further depicts a method 360 for indexing textual medical data. Method 360 may include step 362 of providing a plurality of textual inputs. The plurality of textual inputs may correspond to textual inputs 208. In some aspects, the plurality of textual inputs may include unstructured text, keyword text, structured text, or a combination thereof, as described herein above. In some aspects, the plurality of textual inputs may be obtained from a pathologist, a medical professional, or a patient, or from another machine learning model or artificial intelligence (AI) agent in a workflow.

Method 360 may further include step 364 of receiving a plurality of group level vectors generated by a group level aggregator model. The plurality of group level vectors may correspond to group level vectors 212, and the group level aggregator model may correspond to group level aggregator model 106, 210.

In some aspects, the group level aggregator model may generate the plurality of group level vectors by performing aggregation. In some aspects, the group level aggregator model may generate the plurality of group level vectors by performing aggregation and compression. In some aspects, the group level aggregator model generates the plurality of group level vectors in the form of a vector index containing the plurality of group level vectors in a form suitable for storage and further uses. The vector index may be regularly or continuously updated with new group level vectors generated by the systems and networks described herein. Group level vectors may be split across multiple vector indexes, all mapping to the same patient information source.

The steps of method 340 and method 360 may be combined into a method for indexing a medical dataset including both digital image inputs and textual inputs. For example, slide level aggregator model 210 may perform the step 348 of generating slide level vectors from plurality of tile level vectors 206, and slide level aggregator model 210 may also perform the step 364 of generating slide level vectors from plurality of textual inputs 208. Thus, plurality of slide level vectors 212 includes slide level vectors based on digital image inputs and slide level vectors based on textual inputs, which may further be used and/or added to a vector index by dataset search tool 216.

The steps of method 340 and method 360 may be combined into a method for indexing a medical dataset including both digital image inputs and textual inputs. For example, the method may include the step 348 of receiving group level vectors representing one or more features of interest in entire images from the group level aggregator model, and it may include the step 364 of receiving group level vectors representing one or more features of interest in textual inputs from the same group level aggregator model. The group level aggregator model may also combine vectors representing features of interest from digital image inputs with vectors representing textual inputs that provide data associated with the digital image inputs.

### Querying and Searching

Group level vectors described above may be queried and searched based on a query, regardless of the modality of the query input. Generally, an embedding representing the query input (i.e., a query vector) may be generated and compared to stored group level vectors to quickly identify data relevant to the query.

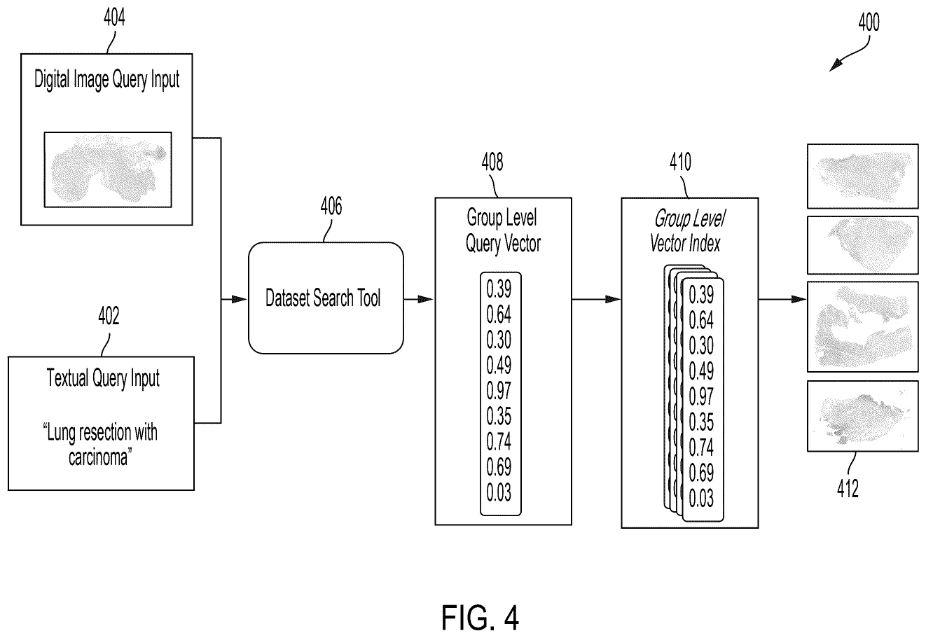

FIG. 4 depicts an exemplary workflow 400 for querying and searching a multimodal medical dataset. Workflow 400 may include providing a textual query input 402 to a dataset search tool 406. In some embodiments, dataset search tool 406 may correspond to dataset search tool 104, 216. Textual query inputs 402 may be provided to a dataset search tool 406 via a server, such as hospital servers 122, research laboratory servers 124, laboratory information servers 126, physician servers 128, and/or clinical trial servers 130. Hospital servers 122, research laboratory servers 124, laboratory information servers 126, physician servers 128, and/or clinical trial servers 130 may also obtain any combination of patient-specific information, such as age, medical history, cancer treatment history, family history, past biopsy, genetic sequencing results, cytology information, etc., and provide it to dataset search tool 406. In some aspects, textual query input 402 may include unstructured text, keyword text, structured text, or a combination thereof, as described above. For example, as depicted in FIG. 4, one exemplary textual query input 402 may include unstructured text “lung resection with carcinoma.”

Workflow 400 may further include providing a digital image query input 404 to dataset search tool 406. Digital image query input 404 may include a digital medical image, such as an image of a cytology specimen, an image of histopathology specimen, a whole slide image, a multiplex immunofluorescent image, a multiplex immunohistochemistry image, a magnetic resonance imaging (MRI) image, a computed tomography (CT) image, an X-ray image, a nuclear medicine imaging (NMI) image, an ultrasound image, a mammography image, an endoscopic image, an angiography image, a confocal microscopy image, a fluorescence in situ hybridization image, an optical coherence tomography image, a bone scan image, a thermography image, an electron microscopy image, or other images supporting detailed visualization and characterization of tissue specimens to evaluate disease mechanisms, progression, and therapeutic response across diverse clinical contexts. For example, as depicted in FIG. 4, one exemplary digital image query input 404 may include a digital image of a slide containing a lung tissue specimen from a patient having carcinoma.

In some aspects, dataset search tool 406 may determine or generate query parameters indicating aspects of the modality, type, or form of query input based on data associated with the source or provider of the query input. For example, where a query input is provided by a user, such as a medical professional, data associated with the user may be used to generate one or more query parameters. The data associated with the user may include bibliographic information (profession, experience level, etc.), past query history (previous query inputs, previously input query parameters, etc.), geographic and setting information (location, clinical setting, etc.), and other data or metadata indicative of the user's input, purpose, or practice in querying the system.

In some aspects, workflow 400 may further include providing (e.g., inputting or automatically determining) one or more query parameters that indicate aspects of the modality, type, or form of query input, as well as contextual information derived from user-specific data. For example, the one or more query parameters may include, in addition to the modality (e.g., textual or digital image), information about the application or diagnostic purpose of the query, the clinical or research setting, and the user's historical interaction data, such as prior queries, search results selected, annotation behavior, or saved preferences. Such contextual information may be used by dataset search tool 406 and/or a foundation model therein to infer user intent and prioritize features or embeddings corresponding to clinically or semantically relevant regions, modalities, or attributes.

In some aspects, query parameters may therefore include information identifying particular semantic or biological features of interest (e.g., tumor boundary, stromal composition, or biomarker expression), disease context (e.g., carcinoma subtype, staging, or treatment status), or linked data modality (e.g., molecular, genetic, or radiologic data) that should be emphasized in the similarity matching process. Where a user's query history or profile indicates a focus on a particular research area or diagnostic task, the system may weight or condition the similarity analysis toward embeddings or group-level vectors representing such regions or modalities.

For example, where the user input includes a digital image of a lung nodule from a carcinoma specimen and the query parameters include an indication of interest in EGFR-expressing nodules, the dataset search tool may identify matching regions based on joint embeddings across image and molecular modalities. In such cases, visual similarity may be determined based on vector-or group-level embeddings of regions expressing similar molecular profiles, while broader whole-image similarity scores may be deprioritized. Matching results may therefore be presented hierarchically: (i) first, regions or groups within images having high correspondence to query embeddings of key regions or modalities; (ii) next, full images containing such regions; and (iii) finally, remaining whole-image matches lacking localized or multimodal correlation, which may be accessible through a “show more” or lower-ranked results option.

In some aspects, the system may also highlight or visually delineate regions of an image corresponding to prioritized vector-or group-level matches to assist user interpretation. For example, when smaller regions of interest within the query image are preferentially matched due to user context or query parameters, such regions may be visually emphasized, while regions matching only general image-level embeddings may be hidden or deemphasized unless specifically requested. In this manner, the system dynamically integrates user-specific, contextual, and multimodal information to refine search precision and relevance.

Dataset search tool 406 may include group level aggregator model 106, 210. Dataset search tool 406 may generate one or more group level query vectors 408 representing one or more features of interest within textual query input 402, digital image query input 404, or a combination thereof, as joint vector representations of features of interest from query input(s). Dataset search tool 406 may then compare the group level query vector 408 to one or more stored group level vectors in group level vector index 410. The stored group level vectors may correspond to group level vectors 212. Dataset search tool may determine a similarity score between the group level query vector 408, representing one or more features of interest from the query input(s), and each stored group level vector within group level vector index 410 representing one or more features of interest from processed medical data.

Based on the similarity score, dataset search tool 406 may determine whether a stored group level vector is relevant to group level query vector 408. A stored group level vector from group level vector index 410 may be relevant to group level query vector 408 if it possesses a similarity score above a specified threshold. A person of ordinary skill in the art would understand that the similarity values and specified threshold may be in any form or value appropriate for the context. For example, the similarity values may be numerical values ranging from 0 to 1, and the specified threshold may be any value between 0 and 1, such as 0.1, 0.2, 0.3, 0.4, 0.5, 0.6, 0.7, 0.8, 0.9, 0.91, 0.92, 0.93, 0.94, 0.95, 0.96, 0.97, 0.98, 0.99, 1, or any value therebetween. The specified threshold may be generated by dataset search tool 406 or it may be received as an input, such as provided by a user.

Workflow 400 may further include outputting a list 412 of source data corresponding to stored group level vectors from vector index 410 determined to be relevant to group level query vector 408. The list 412 may include any form of source data, or it may be filtered, based on an additional query parameter or user input, to only include source data in certain forms or modalities. In some embodiments, list 412 may be output as a list of textual information, a list of digital image information, or a combination thereof. In some aspects, list 412 may be displayed as part of an interactive user interface, allowing a user to click on a result to view further details relating to the result and/or further details on why the result was identified as relevant to the query input.

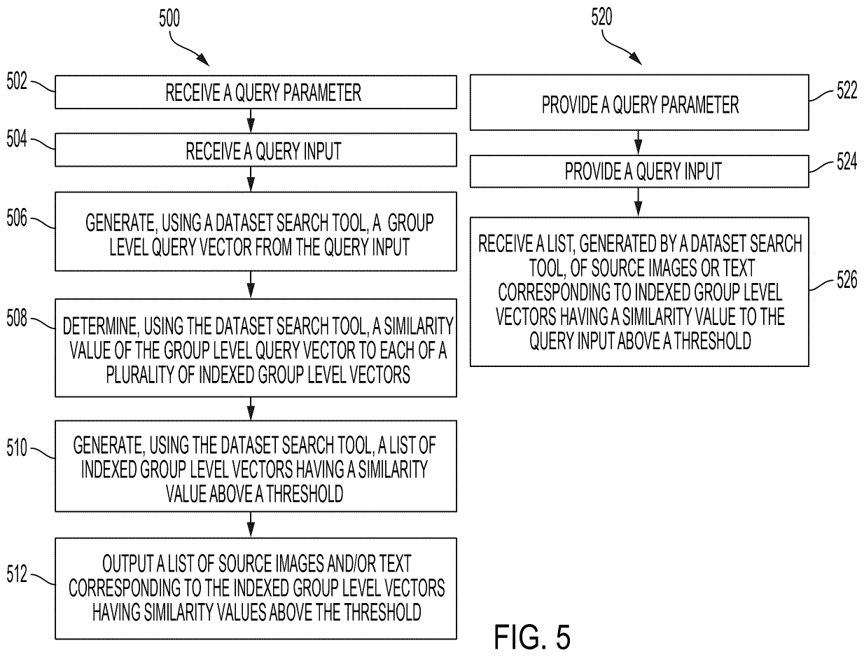

FIG. 5 depicts an exemplary method 500 for querying and searching indexed medical data. Method 500 may include step 504 of receiving a query input corresponding to textual query input 402, digital image query input 404, or a combination thereof. The query input may be received from a user, such as a pathologist, a medical professional, a patient, or a service provider, or from another machine learning model or artificial intelligence (AI) agent in a workflow. In some embodiments, method 500 may optionally include step 502 of receiving a query parameter, prior to receiving query input. The query parameter may indicate aspects of the modality, type, or form of query input. For example, the query parameter may indicate whether the query input is a textual input or a digital image input. In some aspects, the query parameter may indicate further details in addition to type of input. For example, the query parameter may indicate that the query will be a textual input of structured text, such as genetic sequencing data. As another example, the query parameter may indicate that a query will be digital image input of a slide corresponding to a specimen from a particular block or level of tissue. In some aspects, the query parameter may further include parameters for the results to be output. For example, the query parameter may indicate that results to be output should be digital images of specimens from within the same block or level of tissue as a specimen in a slide in a digital image input, or specimens not within the same block or level of tissue.

Method 500 may further include step 506 of generating, using a dataset search tool, a group level query vector from the query input. The dataset search tool may correspond to dataset set tool 104, 216, 406, and the group level query vector may correspond to group level query vector 408.

Method 500 may further include step 508 of determining, using the dataset search tool, a similarity value of the group level query vector to each of a plurality of indexed group level vectors. The plurality of indexed group level vectors may correspond to group level vectors 212 stored in group level vector index 410. In some aspects, an indexed group level vector may be determined to be relevant to the group level query vector if it possesses a similarity score above a specified threshold.

Method 500 may further include step 510 of generating, using the dataset search tool, a list of indexed group level vectors having similarity scores above a specified threshold. The specified threshold may be generated by the dataset search tool, or it may be received as an input, such as provided by a user.

Method 500 may further include step 512 of outputting a list of source images and/or text corresponding to the list of indexed group level vectors having similarity scores above a specified threshold. The list of source images and/or text may correspond to list 412.

FIG. 5 further depicts an exemplary method 520 for querying and searching indexed medical data. Method 520 may include step 524 of providing a query input corresponding to textual query input 402, digital image query input 404, or a combination thereof. The query input may be received from a user, such as a pathologist, a medical professional, a patient, or a service provider, or from another machine learning model or artificial intelligence (AI) agent in a workflow. In some embodiments, method 520 may optionally include step 522 of providing a query parameter, prior to receiving query input. The query parameter may indicate aspects of the modality, type, or form of query input. For example, the query parameter may indicate whether the query input is a textual input or a digital image input. In some aspects, the query parameter may indicate further details in addition to type of input. For example, the query parameter may indicate that the query will be a textual input of structured text, such as genetic sequencing data. As another example, the query parameter may indicate that a query will be digital image input of a slide corresponding to a specimen from a particular block or level of tissue. In some aspects, the query parameter may further include parameters for the results to be output. For example, the query parameter may indicate that results to be output should be digital images of specimens from within the same block or level of tissue as a specimen in a slide in a digital image input, or specimens not within the same block or level of tissue. The query parameter may be provided by a user, such as a pathologist, a medical professional, a patient, or a service provider, or by another machine learning model or artificial intelligence (AI) agent in a workflow.

Method 520 may further include step 526 of receiving a list 412, generated by a dataset search tool, of source images and/or text corresponding to indexed slide level vectors having similarity values above the specified threshold. The dataset search tool may correspond to dataset search tool 104, 216, 406, and the list of source images and/or text may correspond to list 412.

### End-to-end

Provided herein are systems and methods for a complete workflow encompassing all steps of indexing, querying, and searching a medical dataset. The system 100 depicted in FIG. 1 may be configured to perform workflow 200 and workflow 400, depicted in FIG. 2 and FIG. 4, respectively. Dataset search tool 104, 210, 406 may include group level aggregator model 210. Dataset search tool 406 may generate a vector index 410 of group level vectors 212 representing multimodal pathology data, such as various forms and combinations of digital image inputs 202 and/or textual inputs 208. Upon receiving a query input 402, 404, dataset search tool 406 may generate group level query vector 408 and compare group level query vector 408 to plurality of group level vectors 212 stored in vector index 410. Dataset search tool 406 may assign similarity values to each stored group level vector 212 based on a specified threshold, which may be generated by dataset search tool 406, provided by a user, or provided by another machine learning model or artificial intelligence (AI) agent as part of workflow. Dataset search tool 406 may then output a list 412 of source images and/or text corresponding to indexed slide level vectors having similarity values above the specified threshold. List 412 may be output as a list of textual information, a list of digital image information, or a combination thereof. List 412 may be interactive, allowing a user to click on a result to view further details relating to the result and/or further details on why the result was identified as relevant to the query input.

In some embodiments, provided herein is a method combining method 300 and/or method 320 with method 500. The method may include generating plurality of group level vectors 212 based on digital image inputs 202 and/or textual inputs 208, storing plurality of group level vectors 212 in a group level vector index 410, generating a group level query vector 408 based on a query input 402, 404, comparing the group level query vector 40 to each stored group level vector 212 in vector index 410, and outputting a list 412 of source images and/or text corresponding to stored group level vectors 212 having similarity values above a specified threshold.

In some embodiments, provided herein is a method combining method 340 and/or method 360 with method 520. The method may include receiving plurality of group level vectors 212 stored in group level vector index 410 and based on digital image inputs 202 and/or textual inputs 208, providing a query input 402, 404, and receiving a list 412 of source images and/or text corresponding to stored group level vectors 212 having similarity values above a specified threshold.

### Devices

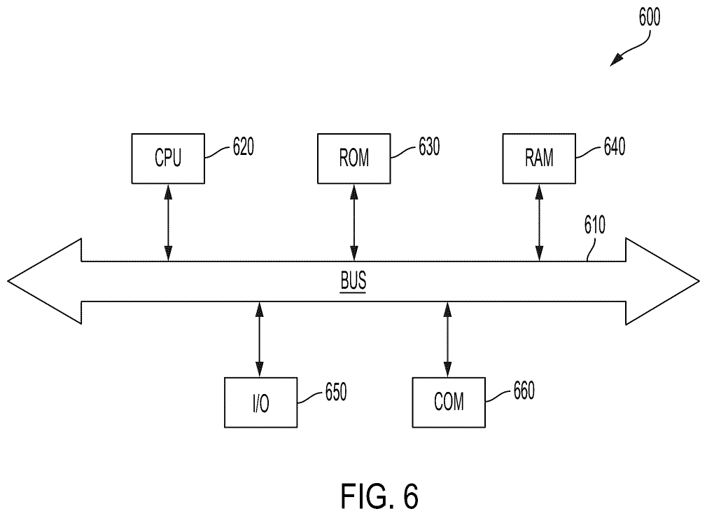

The systems and methods described herein may comprise or utilize a computing device providing hardware architecture and computational infrastructure to support the pathology indexing and retrieval methods and systems described herein. FIG. 6 depicts an exemplary computing device 600 including an underlying hardware platform to implement server systems 102, dataset search tool 104, and associated processing capabilities. Computing device 600 may be configured to execute the machine learning models, vector generation processes, and similarity-based searching operations that may facilitate cross-modal pathology information retrieval across diverse clinical and research environments. Computing device 600 may be deployed as part of distributed computing architectures that may span multiple healthcare institutions and research facilities connected through network 120. The hardware architecture illustrated in computing device 600 may provide the computational foundation for processing large volumes of pathology data while maintaining the performance characteristics needed for real-time query processing and result-retrieval operations.

Computing device 600 may include a central processing unit 620 that may serve as the primary computational component responsible for executing the machine learning algorithms and data processing operations associated with pathology indexing and retrieval functions. Central processing unit 620 may be configured to handle various types of processing tasks including vector generation operations performed by trained foundation model 204, aggregation processes implemented by slide level aggregator model 210, and similarity calculation operations that may compare the slide level query vector 408 against plurality of slide level vectors 212 stored within a slide level vector index 410. In some aspects, central processing unit 620 may comprise specialized processor architectures such as multi-core processors, graphics processing units, or tensor processing units that may be optimized for machine learning computations and parallel processing operations. Central processing unit 620 may also coordinate data flow between different system components and may manage the execution of multiple concurrent processes that may be associated with indexing operations, query processing, and result retrieval functions across diverse pathology datasets.

Computing device 600 may further include a main memory 640 to provide high-speed storage for active data processing operations and temporary storage of computational results during pathology analysis workflows. Main memory 640 may store the machine learning models including trained foundation model 204 and slide level aggregator model 210 during active processing operations, enabling rapid access to model parameters and computational algorithms that may be needed for vector generation and similarity assessment processes. Main memory 640 may also maintain temporary storage of tile level vectors 206, slide level vectors 212, and slide level query vector 408 during active processing operations, facilitating efficient data manipulation and computational operations without requiring frequent access to slower storage systems. Main memory 640 may be configured with sufficient capacity to handle large pathology datasets and may support concurrent processing of multiple query operations that may be submitted by different users accessing dataset search tool 104 through network 120. The high-speed access characteristics of main memory 640 may enable rapid processing of complex machine learning operations while maintaining responsive performance for interactive query and retrieval applications.

Computing device 600 may further include a secondary memory 630 that may provide persistent storage capabilities for maintaining vector index 112, pathology datasets, and associated clinical information that may support long-term data retention and system operation continuity. Secondary memory 630 may store slide level vector index 410 containing the indexed plurality of slide level vectors 212 generated through the indexing processes described in workflow 200, enabling persistent access to comprehensive pathology datasets across system restarts and maintenance operations. Secondary memory 630 may also maintain backup copies of machine learning models, configuration parameters, and system software that may ensure operational continuity and data protection for clinical and research applications. Secondary memory 630 may be configured with storage architectures that may optimize data retrieval performance for similarity-based searching operations while providing sufficient capacity to accommodate growing pathology datasets received from hospital servers 122, research laboratory servers 124, laboratory information servers 126, physician servers 128, and/or clinical trial servers 130. The persistent storage capabilities of secondary memory 630 may enable dataset search tool 104 to maintain comprehensive pathology databases that may support diverse clinical decision-making and research applications across extended operational periods.

Computing device 600 may include a communications interface 660 to enable data transmission and network connectivity between computing device 600 and external systems including healthcare facilities and research institutions. Communications interface 660 may facilitate communication with network 120 that may connect server systems 102 to hospital servers 122, research laboratory servers 124, laboratory information servers 126, physician servers 128, and/or clinical trial servers 130 for pathology data collection and result distribution operations. Communications interface 660 may support multiple communication protocols and network standards that may accommodate diverse institutional systems and data transmission requirements across different healthcare environments. Communications interface 660 may also implement security protocols and data encryption capabilities that may protect sensitive medical information during transmission between computing device 600 and external systems. The network connectivity provided by communications interface 660 may enable real-time data synchronization, continuous dataset updates, and distributed query processing capabilities that may enhance the comprehensiveness and clinical utility of pathology searching and retrieval operations across multiple institutional environments.

Computing device 600 may also include a data communication infrastructure 610 to provide internal connectivity and data transfer capabilities between central processing unit 620, main memory 640, secondary memory 630, and communications interface 660. Data communication infrastructure 610 may comprise bus architectures, interconnect systems, and data pathways that may enable efficient data flow and coordination between different hardware components during pathology processing operations. Data communication infrastructure 610 may be configured to support high-bandwidth data transfers that may be needed for processing large pathology images, transferring vector datasets, and coordinating machine learning computations across different system components. Data communication infrastructure 610 may also provide system control capabilities enabling central processing unit 620 to coordinate operations across different hardware components while maintaining system stability and performance optimization during concurrent processing operations. The internal connectivity provided by data communication infrastructure 610 may ensure that computing device 600 may operate as an integrated system capable of supporting the complex computational requirements associated with pathology indexing, vector generation, similarity assessment, and result retrieval operations across diverse clinical and research applications.

The hardware architecture illustrated in computing device 600 may be scalable and configurable to accommodate varying computational requirements and deployment scenarios across different healthcare and research environments. In some cases, multiple instances of computing device 600 may be deployed in distributed configurations that may provide enhanced processing capacity, redundancy, and geographic distribution of pathology analysis capabilities. Computing device 600 may also be configured with specialized hardware accelerators, additional memory capacity, or enhanced network connectivity that may optimize performance for specific pathology applications or institutional requirements. The modular architecture of computing device 600 may enable healthcare institutions to customize hardware configurations according to their specific pathology data volumes, user populations, and performance requirements while maintaining compatibility with the standardized software systems and machine learning models that may implement the pathology indexing and retrieval capabilities. The flexible deployment options may enable widespread adoption of pathology searching and retrieval technologies across diverse clinical environments while accommodating varying technical infrastructure and resource availability across different healthcare institutions and research facilities.

A number of implementations have been described. Nevertheless, it will be understood that various modifications may be made without departing from the spirit and scope of the disclosure. Accordingly, other implementations are within the scope of the following claims.

1. A method for indexing multimodal dataset, the method comprising:
receiving a multimodal dataset comprising a plurality of digital image inputs and a plurality of textual inputs;
generating, using a group level aggregator model, a first plurality of group level vectors based on the plurality of digital image inputs;
generating, using the group level aggregator model, a second plurality of group level vectors based on the plurality of textual inputs; and
storing the first plurality of group level vectors and the second plurality of group level vectors in an index.

2. The method of claim 1, wherein generating the first plurality of group level vectors comprises:
generating, using a trained foundation model, a plurality of tile level vectors based on each digital image of the plurality of digital image inputs; and
aggregating, using the group level aggregator model, each plurality of tile level vectors to generate the first plurality of group level vectors.

3. The method of claim 2, wherein each group level vector represents one or more features extracted from individual regions within a corresponding digital image of the plurality of digital image inputs.

4. The method of claim 1, wherein the plurality of digital image inputs comprises digital medical images.

5. The method of claim 4, wherein the digital medical images comprise an image of a cytology specimen, an image of histopathology specimen, a whole slide image, a multiplex immunofluorescent image, a multiplex immunohistochemistry image, a magnetic resonance imaging (MRI) image, a computed tomography (CT) image, an X-ray image, a nuclear medicine imaging (NMI) image, an ultrasound image, a mammography image, an endoscopic image, an angiography image, a confocal microscopy image, a fluorescence in situ hybridization image, an optical coherence tomography image, a bone scan image, a thermography image, an electron microscopy image, and/or other images supporting detailed visualization and characterization of tissue specimens to evaluate disease mechanisms, progression, and therapeutic response across diverse clinical contexts.

6. The method of claim 1, wherein the plurality of textual inputs comprises unstructured text, keyword text, structured text, or a combination thereof.

7. The method of claim 6, wherein the unstructured text comprises tabular medical data, diagnosis information, notes regarding sample retrieval and/or preparation, histological details, clinical context involving patient history and other modalities of tests, information specific to staining and markers, morphological observations, transcripts from auditory comments or opinions, references to other tests, references to treatment data, or a combination thereof.

8. The method of claim 6, wherein the keyword text comprises a medical term, a diagnostic code, or a morphological descriptor.

9. The method of claim 6, wherein the structured text comprises genetic sequencing data, genomic data, molecular data, proteomic data, standardized diagnostic reports, coded medical records, or templated clinical forms.

10. The method of claim 1, wherein generating the first plurality of group level vectors and/or the second plurality of group level vectors comprises aggregation.

11. The method of claim 1, wherein generating the first plurality of group level vectors and/or the second plurality of group level vectors comprises aggregation and compression.

12. A system for indexing a multimodal dataset, the system comprising:
at least one memory storing instructions; and
at least one processor configured to execute the instructions to perform operations comprising:
receiving a multimodal dataset comprising a plurality of digital image inputs and a plurality of textual inputs;
generating, using a group level aggregator model, a first plurality of group level vectors based on the plurality of digital image inputs;
generating, using the group level aggregator model, a second plurality of group level vectors based on the plurality of textual inputs; and
storing the first plurality of group level vectors and the second plurality of group level vectors in an index.

13. The system of claim 12, wherein generating the first plurality of group level vectors comprises:
generating, using a trained foundation model, a plurality of tile level vectors based on each digital image of the plurality of digital image inputs; and
aggregating, using the group level aggregator model, each plurality of tile level vectors to generate the first plurality of group level vectors.

14. The system of claim 13, wherein each tile level vector represents one or more features extracted from individual regions within a corresponding digital image of the plurality of digital image inputs.

15. The system of claim 12, wherein the plurality of digital image inputs comprises digital medical images.

16. The system of claim 12, wherein the plurality of textual inputs comprises unstructured text, keyword text, structured text, or a combination thereof.

17. The system of claim 16, wherein:
the unstructured text comprises tabular medical data, diagnosis information, notes regarding sample retrieval and/or preparation, histological details, clinical context involving patient history and other modalities of tests, information specific to staining and markers, morphological observations, transcripts from auditory comments or opinions, references to other tests, references to treatment data, or a combination thereof;
the keyword text comprises a medical term, a diagnostic code, or a morphological descriptor; and/or
the structured text comprises genetic sequencing data, genomic data, molecular data, proteomic data, standardized diagnostic reports, coded medical records, or templated clinical forms.

18. The system of claim 16, wherein generating the first plurality of group level vectors and/or the second plurality of group level vectors comprises aggregation.

19. The system of claim 16, wherein generating the first plurality of group level vectors and/or the second plurality of group level vectors comprises aggregation and compression.

20. A non-transitory computer-readable medium storing instructions that, when executed by a processor, cause the processor to perform a method for indexing a multimodal dataset, the method comprising:
receiving a multimodal dataset comprising a plurality of digital image inputs and a plurality of textual inputs;
generating, using a group level aggregator model, a first plurality of group level vectors based on the plurality of digital image inputs;
generating, using the group level aggregator model, a second plurality of group level vectors based on the plurality of textual inputs; and
storing the first plurality of group level vectors and the second plurality of group level vectors in an index.
